(19) United States
(12) Patent Application Publication
(10) Pub. No.: US 2013/0129732 A1
(43) Pub. Date: May 23, 2013

(54) METHOD FOR PRESERVING FOOD

(71) Applicants: Rebecca Lässker, Arnhem (NL); Edwin Ritzer, Leverkusen (DE)

(72) Inventors: Rebecca Lässker, Arnhem (NL); Edwin Ritzer, Leverkusen (DE)

(73) Assignee: LANXESS Deutschland GmbH, Leverkusen (DE)

(21) Appl. No.: 13/391,826

(22) PCT Filed: Aug. 26, 2010
(86) PCT No.: PCT/EP10/62436

§ 371 Date: Dec. 19, 2012

(30) Foreign Application Priority Data
Aug. 31, 2009 (EP) 09169053.7

(51) Int. Cl. A61K 39/395; C07K 16/46; A61N 5/10

(52) U.S. Cl. CPC A61K 39/39533; A61N 5/10; C07K 16/468
USPC 424/136.1; 600/1; 530/387.3; 435/320.1; 435/328; 435/69.6; 435/254.21; 435/252.33; 435/252.31; 435/252.3; 435/252.35

(57) ABSTRACT

Method for producing microbially stabilized foods characterized in that a food containing a dialkyl dicarbonate is treated by means of electroporation.

The invention relates to a method for producing microbially stabilized foods, in particular drinks, by means of electroporation.

Cold sterilization of drinks is described many times in the literature. It can be effected, firstly, via chemical additives such as preservatives, or by physical methods such as thermal sterilization.

Disadvantages of these methods are frequently an unwanted taste owing to the additives and/or destruction or change of important components, for example, by the thermal methods.

The use of combinations of individual preservatives for drinks is already known for instance, in U.S. Pat. No. 5,738,888 natamycin is used together with dimethyl dicarbonate (DMDC) for sterilizing drinks. In U.S. Pat. No. 6,136,356, a combination of 3 preservatives such as natamycin, sorbate and dimethyl dicarbonate is even required in order to achieve acceptable stabilization results.

Natamycin is an antimycotic and therefore acts principally against fungi. Against fermentation yeasts which are characterized as harmful to drinks owing to dangerous carbon dioxide pressure buildup up to bursting of glass bottles, in contrast, it has an inadequate activity.

In U.S. Pat. No. 6,803,064, Ca-containing drinks are described which can also be preserved, wherein alternatively, one or more preservatives such as sorbates, benzoates or DMDC or pasteurization methods such as PEF can be used. By way of example, however, only a mixture of K-benzoate and K-sorbate in a ratio of 26:7 is used for preservation.

Methods that are gentle to the product, in US-A-2005/0112251, the combination of electroporation methods such as the Pulsed Electric Field (PEF) method in combination with persistent fungicides such as natamycin or sorbate is described.

However, a disadvantage in this method is that the persistent preservatives, particularly sorbic acid, are used in such high concentrations that they exceed the maximum legal amounts for specific foods in many countries. Despite the high usage rates of preservatives, in the case of mould, only a spore seeding of 10² CFU/ml (see Example 2) and in the case of vegetative yeast a microbial seeding of only 10⁴ CFU/ml (see Example 2) was studied. In particular, the weak activity of natamycin against fermentation yeasts can lead to the above-described disadvantages. In WO03/070026, the combination of persistent fungicides such as natamycin or sorbate/sorbic acid in combination with the PEF method is described. Preferably, for example, natamycin is used, which, in addition to the above-described disadvantages, in some countries it is also permitted for the food sector only for specific sausage and cheese varieties such as, for example, in Germany, with respect to its use in human medicine.

As further preferred fungicides, sorbate/sorbic acid is mentioned which, however, is recommended in very high amounts of 500-2000 ppm and also is added at very high dosages in the examples at 800 ppm. The preservatives used in WO03/070026 are therefore either unwanted in drinks or, even in combination with PEF, must be added at very high dosages. The situation is similar for combinations of PEF and, for example, nisin, for certain bacterial strains in Galvez A., et al: “Bacterioncin-based strategies for food biopreservation” International Journal of Microbiology, 2007, pages 51-70.

In US2008/311259, a quite different type of sterilization, high-pressure pasteurization for drinks is described, in which chemical preservatives can also be used in conjunction. A disadvantage in the high-pressure pasteurization is the long treatment time at high pressure which is too time-intensive for drinks in large bottling plants.

The object was therefore to find a method for stabilizing foods, in particular drinks, which does not have the described disadvantages and, furthermore, can be used not only against moulds and yeasts, but also against other microorganisms chiefly occurring in drinks.

A method has now been found for producing microbially stabilized foods, in particular drinks, which is characterized in that a food containing dialkyl dicarbonate is treated by means of electroporation.

A reinforcement in activity has been found which is surprising. Whereas the action of the persistent preservatives such as sorbates or natamycin which also or exclusively react on the membrane of microorganisms may be reinforced by the effect of PEF, DMDC does not act until after passage through the membrane in the interior of the cell by inactivating enzymes. A process which consequently should not be affected by a short-term reversible effect on the membrane. The migration rate of DMDC through the membrane is very much higher than the PEF treatment which is usually in the millisecond range. To this extent a reinforcement in activity was unexpected, as in the case of the preservatives which already develop their activity at the membrane surface (see also “Antimicrobials in Food”, P. M. Davidson, J. N. Sofos and A. L. Branen 2005, pages 49-50 (for sorbates), pages 277 and 280 (for natamycin) and pages 305 and 313 (for DMDC).

Particularly preferably, the dialkyl dicarbonate is a compound of the formula (I)

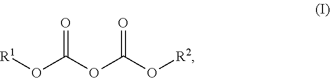

where
R¹ and R² independently of one another are straight-chain or branched C₁-C₈ alkyl, cycloalkyl, C₂-C₈ alkenyl, C₂-C₈ alkynyl or benzyl, each of which may optionally be monosubstituted to polysubstituted, identically or differently, by halogen, nitro, cyano, C₁-C₆ alkoxy and/or dialkylamino, or phenyl, each of which may optionally be monosubstituted to polysubstituted, identically or differently by halogen, nitro, cyano, alkyl, haloalkyl, alkoxy, haloalkoxy, acyl, acyloxy, alkoxycarbonyl and/or carboxyl,
preferably
R¹ and R² independently of one another are straight-chain or branched C₁-C₈ alkyl, C₂-C₈ alkenyl or benzyl,
particularly preferably
R¹ and R² independently of one another are straight-chain or branched C₁-C₅ alkyl, C₃ alkenyl or benzyl,
and very particularly preferably
R¹ and R² independently of one another are methyl, ethyl, isopropyl, tert-butyl, tert-amyl, allyl or benzyl.

Very particularly preferably, dialkyl dicarbonate is dimethyl dicarbonate (DMDC).

The dialkyl dicarbonate is preferably used in an amount of 1 to 300 ppm, in particular from 10 to 260 ppm, based on the food, in particular drink.

The class of substance of dialkyl dicarbonates has the particular property, on contact with corresponding (aqueous) foods, in particular drinks, of hydrolysing into the derived alcohols and carbon dioxide. Depending on the temperature of the drinks during the application, therefore, even after a relatively short time, the actually active substance is already no longer present in the drink. At the customary temperatures of cold packaging of drinks of 0 to 25° C., this is the case after some hours. Improving the activity of dialkyl dicarbonates by various methods or combinations has already been described many times in the patent literature, for example in DE-A-4434314, U.S. Pat. No. 5,738,888, WO 200187096 or US-A-2001046538.

The invention preferably concerns a food that is pumpable at room temperature. Foods, in the context of the invention, are taken to mean substances or products which are intended, or of which, it can be reasonably expected, that they are consumed by humans in the processed, partially processed or unprocessed state. “Foods” also include drinks, chewing gum and also all substances—including water—which are intentionally added to the food during production or processing or preparation thereof.

Particularly preferred foods are drinks, in particular tea-based drinks including green tea, black tea and other tea varieties, and also acidified drinks in particular having a pH≦4.2, carbonated and noncarbonated alcohol-free soft drinks, fruit juices, fruit nectars, fruit juice-containing drinks, fruit preparations, wines, alcohol-free drinks, ciders, ice teas, alcoholic mixed drinks, flavoured waters or sports drinks or isotonic drinks.

It is further preferred to use in conjunction no or further antimicrobially active substances, in particular persistent preservatives, preferably with the exception of natamycin. It is therefore preferred that there is added to the food that is to be treated, in particular drink, further of these substances, in particular additionally at least one further antimicrobially active preservative from the group of the polyene antimycotics such as, for example, nystatin, lucensomycin or amphotericin B, organic acids such as, for example, benzoic acid, sorbic acid, propionic acid or lactic acid, salts of said acids, such as, for example, benzoates, sorbates, propionates or lactates, imidazoles or salts thereof, in particular imazalil, sulphur dioxide, EDTA and lysozyme. Particular preference is given to sodium benzoate and potassium sorbate. Preference, in the case of conjoint use of further antimicrobially active preservatives, is given to using those selected from the group consisting of benzoic acid, sorbic acid, propionic acid, or lactic acid, salts of said acids, such as, for example, benzoates, sorbates, propionates or lactates, imidazoles or salts thereof, in particular imazalil, sulphur dioxide, EDTA and lysozyme.

Very particular preference, in the case of conjoint use of further antimicrobially active preservatives, is given to using those selected from the group consisting of benzoic acid, sorbic acid, propionic acid, or lactic acid, benzoates, in particular sodium benzoate, sorbates, in particular potassium sorbate, propionates and lactates.

Particular preference, in the case of conjoint use of further antimicrobially active preservatives, is given to using those selected from the group consisting of sodium benzoate and potassium sorbate.

Preference is given to the method according to the invention in which no further antimicrobially active substances are added to the food.

Preferably, the food that is to be treated, in particular drink, according to the method according to the invention, contains at least one further antimicrobially active preservative, in particular one of the abovementioned group.

In the case of conjoint use of further antimicrobially active substances, these are used, preferably in an amount of (in the case of salts based on the free acid) 1 to 2000 ppm, in particular from 25 to 500 ppm, preferably 25 to less than 500 ppm, based on the food, in particular think.

The dialkyl dicarbonates, in particular dimethyl dicarbonate, are usually added in liquid form, in portions or continuously to the food, in particular drink. Preference is given to addition of dialkyl dicarbonate preferably proceeding continuously by means of a nozzle, in particular at a pressure of 0.1 to 40 bar, preferably from 0.5 to 40 bar, in particular 10 to 35 bar, over the drink pressure. In particular, DMDC is preferably atomized into the drink stream by means of a metering pump via a heated nozzle. Improvements to corresponding pumps have been described in the patent literature, for example in DE-A-2910328 or in DE-A-2930765. Improvements to the nozzle or to the upstream mixing chamber have been described, for example, in DE-A-1557043. Corresponding metering devices normally consist of storage vessels, electromagnetically operated metering pump, atomizing region and an electronically coupled inductive flow meter, and also of course, intake devices, aeration and temperature control, connections, valves, sensors etc. including all the connecting and controlling electronic elements. The metered outputs of the pumps are normally 0.1 to 20 litres of DMDC per hour.

Dialkyl dicarbonate, in particular DMDC, is added to the food, in particular drink, preferably at a temperature of −5 to 30° C., in particular at 0 to 25° C., particularly preferably at 5 to 22° C.

When further antimicrobially active substances are used in conjunction, they can be added to the drink either separately or together with the dimethyl dicarbonate.

Preferably, the dialkyl dicarbonate compound is added after addition of the other antimicrobially active substance.

On account of the decomposition of the dialkyl dicarbonate compound in the food, in particular drink, it is preferred, after the addition of the dialkyl dicarbonate compound, to add the electroporation as soon as possible thereafter. For instance, the electroporation proceeds preferably at less than 15 minutes after the addition of dialkyl dicarbonate, preferably after less than 5 minutes. The electroporation, in particular the method known as PEF (pulsed electric field), which is also termed high intensity pulsed electric field, in the context of the invention, is a method which is preferably characterized in that pulsed electric fields are allowed to act on the food. Process parameters are primarily the electric field strength and the electrical energy input.

In the PEF electroporation method, food is treated by two electrodes with high-voltage pulses, preferably at field strengths of 0.5 to 100 kV/cm. The PEF method is preferably carried out at −10 to 60° C., in particular at 15 to 25° C. The food in this case is preferably exposed to the energy for less than 1 s, wherein the heating of the food is minimized. In contrast to the thermal methods, the PEF technique is considered to be a better method, since the sensory and physical properties of foods are not affected or are affected only scarcely thereby.

The high field strength in the PEF technique is generally achieved in that a large part of the energy is stored in a condenser bank of a direct current power supply, which is then discharged in the form of high-voltage pulses.

Examples of such PEF apparatuses, and the description of their mode of operation can be found, for example, in DE-A-3413583.

In the electroporation, in particular the PEF method, according to the invention, preferably energy densities of 1 to 1000 J/ml of food, in particular drink, are used, preferably 15 to 200 J/ml. For the method according to the invention field strengths of preferably 0.5 to 100 kV/cm, in particular 3 to 50 kV/cm are advantageous. The frequency of the electrical pulses is preferably 10 to 800 Hz, preferably 60 to 500 Hz.

The length of the pulse (pulse width) is preferably 1 to 100 μs, in particular 5 to 50 μs.

The energy density is defined as follows.

$$Energy density = \frac{3600 \times efficiency \times absorbed power [ W ]}{flow rate [ l \text{/} h ] \times 1000}$$

The efficiency is, for example, in the Elcrack® apparatus used in the examples, approximately 85%. The absorbed power in watts was read off from the PEF apparatus during the treatment method. The flow rate was 70 l/h.

The treated food, in particular drink, is generally warmed by approximately 1 to 20° C., depending on the electrical energy input.

Particularly preferably, the method according to the invention is used against the following strains: bacteria (e.g. Bacillus spp., Lactobacillus spp., Leuconostoc spp., Acetobacter spp., Gluconacetobacter spp., Alicyclobacillus spp.), yeasts (e.g. Saccharomyces spp., Zygosaccharomyces spp., Trichoderma spp., Candida spp., Brettanomyces spp., Pichia spp.) and moulds (e.g. Penicillium spp., Byssochlamys spp., Aspergillus spp., Fusarium spp.). Surprisingly, it has been found that the method according to the invention leads to a marked improvement in sterilization, the consequence of which is that either higher initial microbial contamination of drinks can be combated using the same amounts of dialkyl dicarbonate optionally in combination with persistent preservatives, or, at usual microbial contamination, small amounts will still be sufficient.

The method according to the invention therefore has the advantage of effectively combating microorganisms without needing persistent preservatives, or without needing high amounts, and possibly absolutely impermissible amounts, of persistent preservatives.

In addition, it has been found that bacterial spores and bacteria which are slime formers and therefore are able to produce biofilms which are substantial problems in the food industry (e.g. Lactobacillus frigidus) can be combated effectively. In addition, endospore-forming bacteria, for example Bacillus subtilis have been studied. Endospores are survival forms of bacteria (e.g. Bacillus, Clostridium) which, in comparison with vegetative bacterial cells, can generally survive 100° C. for several hours and are not reliably destroyed until after heating to 120° C. for several minutes. The method according to the invention could even be used successfully against these microorganisms.

### EXAMPLES

In the examples hereinafter, the Elcrack® apparatus of the Deutsche Institute für Lebensmitteltechnik e. V. [German Institute for food technology] was used for the PEF treatment.

The treatment cell of the apparatus had a diameter of 5 mm here and an electrode separation of 7 mm. The treatment proceeded at differing field strengths and energy densities, wherein the pulse width was 20 μs and the pulse frequency 400 Hz.

In the examples hereinafter, in each case clear apple juice was admixed with stirring by means of a propeller agitator with a microbial or spore suspension, then after approximately 3 minutes, optionally further antimicrobially active substances, and thereafter after approximately 2 minutes, DMDC. The microbial or spore suspension was added in such a manner that a defined amount of cells, generally 10³ to 10⁵ CFU/ml were present in the apple juice.

The apple juice was then treated by PEF (using the above-described Elcrack® apparatus). As further microbially active substances, sodium benzoate or potassium sorbate were used.

At differing energy densities and field strengths, the temperatures of the finally treated drinks reported in the table were determined.

As a measure for microbial reduction, the median logarithmic cell count reduction (MLC) was determined. In this case the logarithm of the surviving cell count is subtracted from the logarithm of the initial cell count. The higher the MLC value, the higher the microbial reduction and the better the activity is.

V is a comparative example

The microorganisms used were the following.

A: Bacillilus subtilis DSM 347 (ATCC 6633)
B: Penicillium roqueforti DSM 1079 (ATCC 34908)
C: Lactobacillus frigidus DSM 6235
D: Saccharomyces cervical DSM 70449 (ATCC 18824)

TABLE

| | | | Antimicrobially | Field | Energy | Temperature | |
|---|---|---|---|---|---|---|---|
| | | | active substance/ | strength | density | after | MLC/ |
| Example | Microorganism | [CFU/ml] | amount in ppm 1) | [kV/cm] | [J/ml] | treatment | Time 2) |
| A1 | Bacillus subtilis | 10³ | DMDC/250 | 35 | 111 | 36° C. | 2.2/24 h |
| A2 | ″ | 10³ | DMDC/250 | 35 | 110 | 36° C. | 3.0/24 h |
| | | | benzoate/150 | | | | |
| A3 | ″ | 10³ | DMDC/250 | 35 | 116 | 36° C. | 2.1/24 h |
| | | | sorbate/300 | | | | |
| A4 | ″ | 10³ | DMDC/250 | 15 | 23 | 25° C. | 1.4/24 h |
| A5 | ″ | 10³ | DMDC/250 | 35 | 111 | 36° C. | 3.0/1 week |
| VA1 | ″ | 10³ | benzoate/150 | 35 | 111 | 36° C. | 2.0/24 h |
| VA2 | ″ | 10³ | sorbate/300 | 35 | 116 | 36° C. | 0.5/24 h |
| VA3 | ″ | 10³ | —/— | 35 | 111 | 36° C. | 2.7/1 week |
| VA4 | ″ | 10³ | —/— | — | — | 21° C. | none; totally |
| | | | | | | | microbially |
| | | | | | | | contaminated |
| B1 | Penicillium roqueforti | 10⁵ | DMDC/250 | 30 | 94 | 32° C. | at least. 7/24 h |
| B2 | ″ | 10⁵ | DMDC/250 | 30 | 103 | 32° C. | at least 7/24 h |
| | | | benzoate/150 | | | | |
| B3 | ″ | 10⁵ | DMDC/250 | 30 | 104 | 32° C. | at least 7/24 h |
| | | | sorbate/300 | | | | |
| B4 | ″ | ″ | DMDC/250 | 30 | 104 | 32° C. | at least 7/4 |
| | | | sorbate/300 | | | | weeks |
| B5 | ″ | ″ | DMDC/250 | 30 | 94 | 32° C. | at least 7/4 |
| | | | | | | | weeks |
| VB1 | ″ | 10⁵ | DMDC/250 | — | — | 21° C. | 6.4/24 h |
| VB2 | ″ | 10⁵ | —/— | 30 | 98 | 32° C. | none; heavy |
| | | | | | | | microbial |
| | | | | | | | growth/24 h |
| VB3 | ″ | ″ | benzoate/150 | 30 | 99 | 32° C. | none; heavy |
| | | | | | | | microbial |
| | | | | | | | growth/24 h |
| VB4 | ″ | ″ | sorbate/300 | 30 | 104 | 32° C. | none; heavy |
| | | | | | | | microbial |
| | | | | | | | growth/24 h |
| VB5 | Penicillium roqueforti | 10⁵ | —/— | 30 | 98 | 32° C. | none; heavy |
| | | | | | | | microbial |
| | | | | | | | growth/4 |
| | | | | | | | weeks |
| C1 | Lactobacillus frigidus | 10⁵ | DMDC/250 | 30 | 93 | 32° C. | 3.6/1 week |
| C2 | ″ | ″ | DMDC/250 | 30 | 92 | 32° C. | 3.5/1 week |
| | | | benzoate/150 | | | | |
| C3 | ″ | ″ | DMDC/250 | 30 | 91 | 32° C. | 3.6/1 week |
| | | | sorbate/300 | | | | |
| VC1 | ″ | ″ | DMDC/250 | — | — | 21° C. | 2.3/1 week |
| VC2 | ″ | ″ | benzoate/150 | 30 | 88 | 32° C. | 2.8/1 week |
| VC3 | ″ | ″ | sorbate/300 | 30 | 91 | 32° C. | 3.0/1 week |
| VC4 | ″ | ″ | —/— | 30 | 83 | 32° C. | 3.4/1 week |
| VC5 | ″ | ″ | —/— | — | — | 21° C. | 2.3/1 week |
| D1 | Saccharomyces cerivisiae | 10⁵ | DMDC/250 | 5 | 3 | 22° C. | min 7/24 h |
| D2 | ″ | ″ | DMDC/250 | 5 | 3 | 22° C. | min 7/24 h |
| | | | benzoate/150 | | | | |
| D3 | ″ | ″ | DMDC/250 | 5 | 3 | 22° C. | min 7/24 h |
| | | | sorbate/300 | | | | |
| VD1 | ″ | ″ | —/— | 5 | 2 | 22° C. | none; heavy |
| | | | | | | | microbial |
| | | | | | | | growth/24 h |
| VD2 | ″ | ″ | benzoate/150 | 5 | 3 | 22° C. | none; heavy |
| | | | | | | | microbial |
| | | | | | | | growth/24 h |
| VD3 | ″ | ″ | sorbate/300 | 5 | 6 | 22° C. | none; heavy |
| | | | | | | | microbial |
| | | | | | | | growth/24 h |

1) Amount of sorbate and benzoate is reported in each case as free acid.
2) For the value “at least 7” the following applies: The microbial seeding for the vegetative cells was 10⁵ CPU/ml. For the microbiological studies 100 ml of the sample were membrane filtered and analysed, which then results in a value of at least 7, since nothing had grown.

What is claimed is:

1. Method for producing microbially stabilized foods, characterized in that a food containing a dialkyl dicarbonate is treated by means of electroporation.

2. Method according to claim 1, characterized in that the dialkyl dicarbonate is a compound of the formula (I) 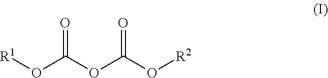
where
R¹ and R² independently of one another are straight-chain or branched C₁-C₈ alkyl, cycloalkyl, C₂-C₈ alkenyl, C₂-C₈ alkynyl or benzyl, each of which may optionally be monosubstituted to polysubstituted, identically or differently, by halogen; nitro; cyano; C₁-C₆ alkoxy; dialkylamino; or phenyl, each of which may optionally be monosubstituted to polysubstituted, identically or differently by halogen; nitro; cyano; alkyl; haloalkyl; alkoxy; haloalkoxy; acyl; acyloxy; alkoxycarbonyl; carboxyl,
preferably
R¹ and R² independently of one another are straight-chain or branched C₁-C₈ alkyl, C₂-C₈ alkenyl or benzyl,
particularly preferably
R¹ and R² independently of one another are straight-chain or branched C₁-C₅ alkyl, C₃ alkenyl or benzyl,
and very particularly preferably
R¹ and R² independently of one another are methyl, ethyl, isopropyl, tert-butyl, tert-amyl, allyl or benzyl.

3. Method according to claim 1, characterized in that the dialkyl dicarbonate is dimethyl dicarbonate.

4. Method according to claim 1, characterized in that the food is a drink.

5. Method according to claim 1, characterized in that the food is tea-based drinks including green tea, black tea and other tea varieties, and also acidified drinks having a pH≦4.2, carbonated and noncarbonated alcohol-free soft drinks, fruit juices, fruit nectars, fruit juice-containing drinks, fruit preparations, wines, alcohol-free drinks, ciders, ice teas, alcoholic mixed drinks, flavoured waters, sports drinks or isotonic drinks.

6. Method according to claim 1, characterized in that a further antimicrobially active preservative is additionally added to the food that is to be treated.

7. Method according to claim 1, characterized in that at least one further antimicrobially active preservative from the group benzoic acid, benzoates, sorbic acid, sorbates, propionic acid, propionates, nisin, sulphur dioxide, EDTA and lysozyme is additionally added to the food that is to be treated.

8. Method according to claim 1, characterized in that the energy density introduced into the food drink by means of electroporation is 1 to 1000 J/ml.

9. Method according to claim 1, characterized in that, as electroporation, the pulsed electric field method is used.

10. Method according to claim 1, characterized in that the dialkyl dicarbonate is added in an amount of 10 to 250 ppm, based on the food, in particular drink.
